(19) United States
(12) Patent Application Publication
(10) Pub. No.: US 2026/0321350 A1
(43) Pub. Date: Oct. 15, 2026

(54) USING PREDICTED IONIC CURRENT SIGNALS GENERATED DURING NANOPORE TRANSLOCATION TO CLASSIFY PROTEINS

(71) Applicant: UNIVERSITY OF WASHINGTON, Seattle, WA (US)

(72) Inventors: Melissa QUEEN, Seattle, WA (US); Jeffrey Matthew NIVALA, Seattle, WA (US); Daphne KONTOGIORGOS-HEINTZ, Seattle, WA (US)

(73) Assignee: UNIVERSITY OF WASHINGTON, Seattle, WA (US)

(21) Appl. No.: 19/484,813

(22) PCT Filed: May 17, 2024
(86) PCT No.: PCT/US2024/030099

§ 371 Date: Nov. 14, 2025

Related U.S. Application Data
(60) Provisional application No. 63/467,557, filed on May 18, 2023.
(60) Provisional application No. 63/467,745, filed on May 19, 2023.
(60) Provisional application No. 63/542,154, filed on Oct. 3, 2023.

(51) Int. Cl. G16B 40/10; G06N 3/0464; G06N 3/0895; G16B 30/00; G16B 40/20

(52) U.S. Cl. CPC G16B 40/10; G06N 3/0464; G06N 3/0895; G16B 30/00; G16B 40/20

(57) ABSTRACT

In some embodiments, a computer-implemented method of training a classifier model for classifying a protein as a specific protein from a protein library is provided. A computing system generates a template squiggle for each protein in the protein library. The computing system trains a classifier model based on the template squiggles, and stores the classifier model in a model data store. In some embodiments, the computing system also receives a squiggle representing a signal generated by passing the protein through a nanopore, and classifies the protein by using the trained classifier model to classify the squiggle.

### CROSS-REFERENCES TO RELATED APPLICATIONS

This application claims the benefit of Provisional Application No. 63/542,154, filed Oct. 3, 2023; Provisional Application No. 63/467,745, filed May 19, 2023; and Provisional Application No. 63/467,557, filed May 18, 2023, the entire disclosures of which are hereby incorporated by reference herein for all purposes.

### STATEMENT OF GOVERNMENT LICENSE RIGHTS

This invention was made with government support under Grant No. 1R01HG012545, awarded by the National Institutes of Health. The government has certain rights in the invention.

### BACKGROUND

Proteins play a fundamental role in virtually all biological processes. The Central Dogma of molecular biology decrees that genetic information flows from DNA to RNA to protein. The current accepted model accounts for over 20,000 of these protein-coding genes among the human genome. The collection of these proteins is referred to as the human proteome. Even after translation from RNA to protein, there exist mechanisms within the human body to modify these proteins further into distinct proteoforms, thereby expanding an ever-growing catalog of human proteins.

With such a large dictionary of proteins, each with unique structures and functions, a natural investigation would be finding a system to classify them. Protein fingerprinting is a process of uniquely identifying and/or characterizing individual proteins based on their distinct structural and functional properties. Enabling such a scheme over the vast human proteome would be transformative for research in medicine and healthcare: By accurately identifying proteins, especially those associated with a certain disease or genetic mutation, clinicians can expand their breadth and gain stronger confidence for medical treatments, even potentially providing precise medicine on an individual level. Additionally, understanding the proteins involved in all stages of the disease pipeline can facilitate the development of targeted drugs. Categorizing proteins post-translationally provides rich insight into their structure, function, and interaction.

Current fingerprinting technology exists, but there are some notable challenges, mostly in that the existing techniques tend to lack precision and/or require domain knowledge a priori. For example, antibody arrays are a standard tool used to detect and quantify the presence of multiple proteins simultaneously within a biological sample, but require researcher's prior knowledge about the proteins of interest. Scientists must know which proteins they expect to measure to select appropriate capture agents (e.g. antibodies) to design the array.

Mass Spectrometry (MS) is another existing technique to identify and quantify proteins based on their mass-to-charge ratio. Although the results are often accurate, the process is inherently destructive, degenerating the protein of interest as information about it is obtained Other models for protein fingerprinting also exist, but can be expensive, time-consuming, and often inaccurate. Given the lack of standard technology, techniques are desired that can fingerprint human proteins cheaply and accurately, without relying on domain knowledge and without destroying the protein in the process.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of training a classifier model for classifying a protein as a specific protein from a protein library is provided. A computing system generates a template squiggle for each protein in the protein library. The computing system trains a classifier model based on the template squiggles, and stores the classifier model in a model data store. In some embodiments, the computing system also receives a squiggle representing a signal generated by passing the protein through a nanopore, and classifies the protein by using the trained classifier model to classify the squiggle.

In some embodiments, a computer-implemented method of classifying a protein as a specific protein from a protein library is provided. A computing system receives a squiggle representing a signal generated by passing the protein through a nanopore, and classifies the protein by using a trained classifier model to classify the squiggle. In some embodiments, the trained classifier model was trained based on template squiggles generated for each protein in the protein library.

In some embodiments, a non-transitory computer-readable medium having computer-executable instructions stored thereon is provided. The instructions, in response to execution by one or more processors of a computing system, cause the computing system to perform actions of a method as described above. In some embodiments, a computing system having such a computer-readable medium is provided.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a high-level schematic illustration of the analysis of a protein using a nanopore according to various aspects of the present disclosure.

FIG. 2 is a schematic illustration of a system for nanopore-based analysis according to various aspects of the present disclosure.

FIG. 3 is a schematic illustration of a non-limiting example embodiment of a flow cell according to various aspects of the present disclosure.

FIG. 4 is a block diagram that illustrates aspects of a non-limiting example embodiment of a classification computing system according to various aspects of the present disclosure.

FIG. 5 is a flowchart that illustrates a non-limiting example embodiment of a method for determining a template squiggle based on a sequence, according to various aspects of the present disclosure.

FIG. 6A-FIG. 6B are a flowchart that illustrates a non-limiting example embodiment of a method of using a machine learning model to classify proteins based on nanopore signals, according to various aspects of the present disclosure.

FIG. 7 is a flowchart that illustrates a non-limiting example embodiment of a method of using a nearest-neighbor model to classify proteins based on nanopore signals, according to various aspects of the present disclosure.

### DETAILED DESCRIPTION

As noted above, the human proteome consists of tens of thousands of proteins produced from sequences translated from the human genome. Further, each of these proteins can be modified post-translationally to create an even larger set of unique proteoforms. With such a massive catalog, techniques that accurately and inexpensively fingerprint proteins with single-molecule resolution in real-time would have a transformative impact on biology, medicine, and healthcare.

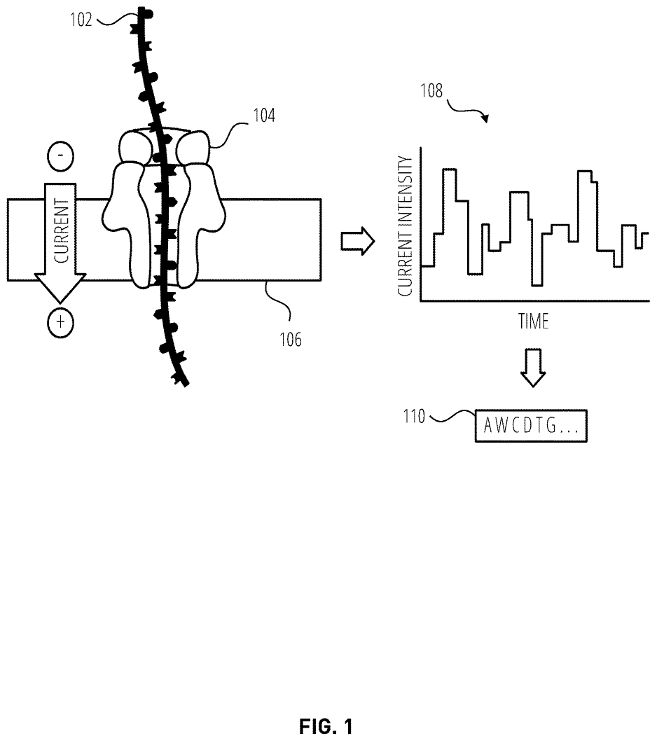

To this end, embodiments of the present disclosure provide techniques for analyzing the output of nanopore sensor technology to identify proteins with single-molecule resolution. FIG. 1 is a high-level schematic illustration of the analysis of a protein using a nanopore according to various aspects of the present disclosure. A “nanopore,” such as nanopore 104, is a microscopic trans-membrane hole embedded within an electro-resistant membrane 106. Nanopores work by electrically examining proteins, such as sample protein 102, at the molecular scale as they diffuse through the nanopore 104. In some embodiments, the nanopore 104 may be associated with a motor protein that helps control the passage of the sample protein 102 through the membrane 106.

When the nanopore 104 is unblocked, current is free to pass through at a constant intensity. This current is disturbed as the sample protein 102 passes through the nanopore 104. The amount of interference depends on the relative size, structure, and charge of the current subsection of the sample protein 102 within the nanopore 104. So, each different sample protein 102 induces a unique alteration of the current, which reflects a sequence of residues of the protein. By feeding the whole sample protein 102 through the nanopore 104, measuring current at equally spaced time steps, we obtain a unique current-time signature of the sample protein 102. This signal, referred to herein as a “squiggle” and illustrated in FIG. 1 as squiggle 108, acts as a protein-specific molecular fingerprint and provides the ability to generate a classification 110 of the sample protein 102.

In embodiments of the present disclosure, squiggles are provided to machine learning models to classify proteins. Two non-limiting example machine learning model architectures described in the present disclosure are a Convolutional Neural Network (CNN) model and a Nearest Neighbors (NN) model. CNNs are a type of artificial neural network that use distinct filters and pooling to identify unique “features” in the input. NNs are a non-parametric model that compares an unclassified protein with a subset of classified proteins, invoking a distance metric to choose the group with the lowest distance among its nearest neighbors.

It is known that training of machine learning models uses training data that represents the data to be analyzed. Given the size of the human proteome, it is impractical to expect that an adequate number of squiggles may be collected experimentally to be used to successfully train either a CNN model or a NN model. Accordingly, the present disclosure also provides techniques for using known sequences associated with proteins to predict squiggles that would be generated if the proteins were to be analyzed by a nanopore, and to use the predicted squiggles to generate training data that can successfully train either a CNN model or a NN model.

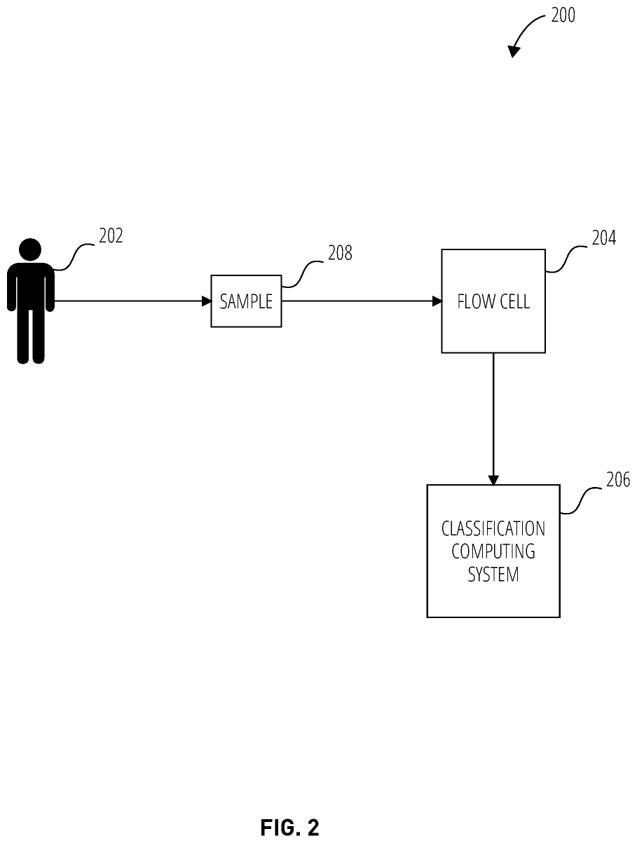

FIG. 2 is a schematic illustration of a system for nanopore-based analysis according to various aspects of the present disclosure. As shown, in the system 200, a sample 208 is obtained from a subject 202 using known techniques. The sample 208 may be a tissue biopsy, a swab, a blood sample, or any other suitable type of sample 208. The sample 208 is prepared (e.g., combined with one or more buffers, enzymes, etc.), and the prepared sample 208 is provided to a flow cell 204 of a sequencing device. One non-limiting example of a sequencing device is a MinION sequencing device provided by Oxford Nanopore Technologies plc. Some non-limiting examples of devices for implementing a flow cell 204 are a Flongle Flow Cell, a MinION Flow Cell, and the PromethION Flow Cell, each also provided by Oxford Nanopore Technologies plc. The flow cell 204 generates signals based on interactions between the sample 208 and the nanopores of the flow cell 204, and provides the signals to the classification computing system 206 for analysis.

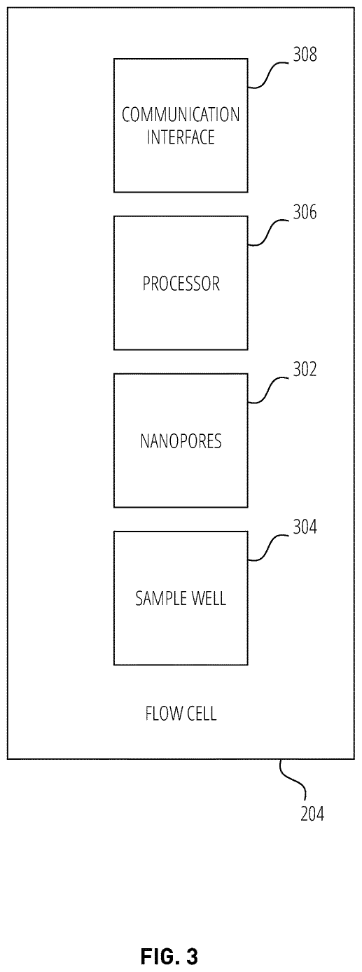

FIG. 3 is a schematic illustration of a non-limiting example embodiment of a flow cell according to various aspects of the present disclosure. As shown, the flow cell 204 includes a sample well 304, a plurality of nanopores 302, a processor 306, and a communication interface 308. The sample well 304 is configured to accept the sample 208 (e.g., to receive drops of sample 208 from a pipette) and to provide the sample 208 to the plurality of nanopores 302. The processor 306 is configured to control a voltage applied to the plurality of nanopores 302 and to read signals generated by the nanopores 302. In some embodiments, the processor 306 may also be configured to segment the signals generated by the nanopores 302 into a plurality of segmented events, each segmented event representing an interaction of a molecule with a nanopore 302 of the plurality of nanopores 302. In some embodiments, the processor 306 may be configured to perform base-calling (determining an identity of an amino acid represented by one or more segmented events). In some embodiments, the communication interface 308 is configured to transmit the signals detected by the processor 306, squiggles representing the signals detected by the processor 306, the segmented events, and/or the base-calling results to another device, such as the classification computing system 206, using a wired or wireless network, a USB connection, or any other suitable communication technique. In some embodiments, the processor 306, communication interface 308, and potentially other components (such as a computer-readable medium) may be implemented on an ASIC or FPGA that is part of the flow cell 204.

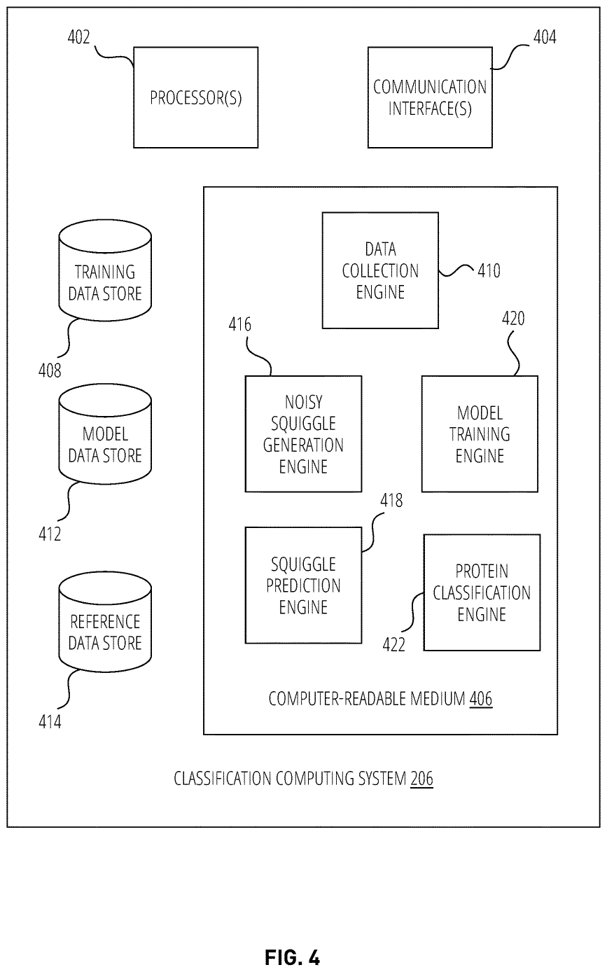

FIG. 4 is a block diagram that illustrates aspects of a non-limiting example embodiment of a classification computing system according to various aspects of the present disclosure. The illustrated classification computing system 206 may be implemented by any computing device or collection of computing devices, including but not limited to a desktop computing device, a laptop computing device, a mobile computing device, a server computing device, a computing device of a cloud computing system, and/or combinations thereof. The classification computing system 206 is configured to train and/or use machine learning models that can classify a protein based on a squiggle representing a signal generated by a nanopore.

As shown, the classification computing system 206 includes one or more processors 402, one or more communication interfaces 404, a training data store 408, a model data store 412, a reference data store 414, and a computer-readable medium 406.

In some embodiments, the processors 402 may include any suitable type of general-purpose computer processor. In some embodiments, the processors 402 may include one or more special-purpose computer processors or AI accelerators optimized for specific computing tasks, including but not limited to graphical processing units (GPUs), vision processing units (VPUs), and tensor processing units (TPUs).

In some embodiments, the communication interfaces 404 include one or more hardware and or software interfaces suitable for providing communication links between components. The communication interfaces 404 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As shown, the computer-readable medium 406 has stored thereon logic that, in response to execution by the one or more processors 402, cause the classification computing system 206 to provide a data collection engine 410, a model training engine 420, a noisy squiggle generation engine 416, a squiggle prediction engine 418, and a protein classification engine 422.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

In some embodiments, the data collection engine 410 is configured to receive squiggles from nanopores. In some embodiments, the protein classification engine 422 is configured to use trained machine learning models stored in the model data store 412 to classify the squiggles received by the data collection engine 410. In some embodiments, the model training engine 420 is configured to generate training data to be stored in the training data store 408, and to use the training data to train machine learning models. In some embodiments, the squiggle prediction engine 418 is configured to generate template squiggles based on sequence information stored in the reference data store 414, and the template squiggles may be used by the model training engine 420 as the training data or to generate the training data. In some embodiments, the noisy squiggle generation engine 416 is configured to generate additional training data to be stored in the training data store 408 based on the template squiggles.

Further description of the configuration of each of these components is provided below.

As used herein, “engine” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

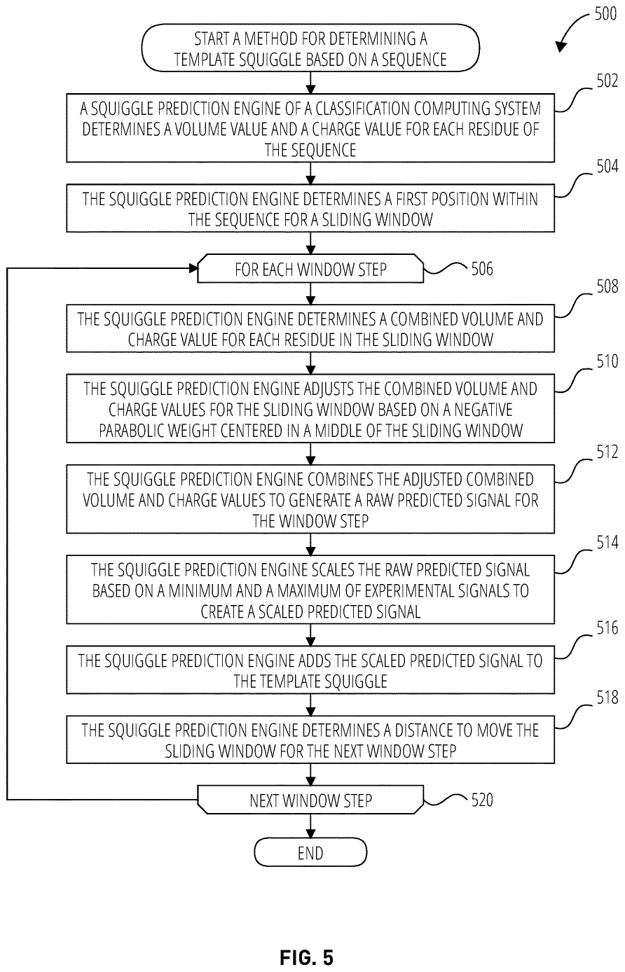

FIG. 5 is a flowchart that illustrates a non-limiting example embodiment of a method for determining a template squiggle based on a sequence, according to various aspects of the present disclosure. As discussed above, one of the challenges in using machine learning models to classify proteins in a protein library such as the human proteome is that the vast number of different proteins makes it impractical to collect training data representing every protein. However, the sequence of residues for these proteins are known. Accordingly, the method 500 illustrated in FIG. 5 may be used to predict a template squiggle for a protein using the known sequences, and this template squiggle may be used to train machine learning models in lieu of experimentally generated squiggles. The method 500 assumes that the sequence for the protein is known, and has been retrieved from the reference data store 414. The sequence may be represented by a vector of amino acid (“aa”) residue values:

Seq=[aa₁, aa₂, . . . , aaₙ].

From a start block, the method 500 proceeds to block 502, where a squiggle prediction engine 418 of a classification computing system 206 determines a volume value and a charge value for each residue of the sequence. In some embodiments, the volumes and charges of each residue are known based on the physical properties of the residues, and/or may be experimentally determined or verified. In some embodiments, the known volume values and charge values may be retrieved from the reference data store 414, and/or may be based on an assumed pH value, such as a pH value of 7.6, where a histidine residue is assumed to be neutral.

At block 504, the squiggle prediction engine 418 determines a first position within the sequence for a sliding window. The sliding window represents a number of residues that may be present within the nanopore at once and/or that otherwise collectively have an effect on the signal generated by the nanopore. Any suitable size may be used for the sliding window. It has previously been determined that 20 residues may be present in or otherwise affect the signal generated by the nanopore, and so in some embodiments, the sliding window may have a size of 20 residues. In some embodiments, other sizes may be used for the sliding window, including but not limited to a size selected from a range of 14-26 residues. In some embodiments, the size of the sliding window may be determined experimentally or via modeling of the structure of the nanopore 104 and/or one or more motor proteins associated with the nanopore 104. In some embodiments, instead of being constant, the size of the sliding window may be determined as a function of the sequence within a broader window. In some embodiments, the squiggle prediction engine 418 may use the first residue of the sequence as the first position for the sliding window. In some embodiments, the squiggle prediction engine 418 may use a different residue of the sequence as the first position for the sliding window, such as a position other than the start of the sequence that is known to be a start of a distinctive area of the sequence.

The method 500 then proceeds to a for-loop defined between a for-loop start block 506 and a for-loop end block 520. Since the content of the sliding window represents the residues that are affecting the signal generated by the nanopore at a given time, each iteration of the for-loop processes the content of the sliding window to generate a single data point of the template squiggle before moving the sliding window to generate the next data point of the template squiggle.

Accordingly, from for-loop start block 506, the method 500 proceeds to block 508, where the squiggle prediction engine 418 determines a combined volume and charge value for each residue in the sliding window. In some embodiments, the volume value and the charge value may each be weighted prior to being combined in order to improve the accuracy of the results. The combination of weighted volume value and weighted charge value for each position j within the sliding window starting at index i in the sequence may be expressed as:

$${V}_{c}^{⋆} Volume ( {aa}_{i + j} ) + {C}_{c}^{⋆} Charge ( {aa}_{i + j} ) ,$$

where V_{c} is the weight to be applied to the volume value and Ce is the weight to be applied to the charge value. As a non-limiting example, the volume values may each be multiplied by a weight of 4×10⁻⁴ and the charge values may each be multiplied by a weight of 1.2×10⁻⁴ prior to being added together for each residue. These non-limiting example weights were determined empirically to minimize the dynamic time warping (DTW) distance scores of a set of training data, but in other embodiments, other appropriate weights may be used.

At block 510, the squiggle prediction engine 418 adjusts the combined volume and charge values for the sliding window based on a negative parabolic weight centered in a middle of the sliding window. The residues closer to the center of the sliding window are also closer to the middle of the nanopore, and so tend to have a greater impact on the signal generated by the nanopore. Accordingly, the negative parabolic weight reduces the contribution of the values closer to the start and end of the sliding window compared to the contribution of the values closer to the center of the sliding window. The negative parabolic weight is a non-limiting example of an adjustment to be applied to the combined volume and charge values for the sliding window to reflect the relative effect of the residues on the signal produced by the nanopore by virtue of their relative positions in the nanopore. In some embodiments, other types of weights may be applied across the sliding window. For example, the definition of the weights may be a function of the sequence within the sliding window itself.

At block 512, the squiggle prediction engine 418 combines the adjusted combined volume and charge values to generate a raw predicted signal for the window step. In some embodiments, a weight vector that includes the negative parabolic weights may be precomputed, and blocks 510 and 512 may be performed at the same time by computing a dot product of the weight vector and the vector of the combined volume and charge values. This operation may be represented as follows:

$${[ P W ]}_{i} = - {aj}^{2} + bj - c , for 1 \leq j \leq 20$$

$${S}_{i} = P W \cdot {X}_{i}$$

wherein [PW]ᵢ represents the weight vector for the sliding window location i, Xᵢ represents the vector of adjusted combined volume and charge values for the sliding window location i, and Sᵢ is a scalar value representing the raw predicted signal for the sliding window location i.

At block 514, the squiggle prediction engine 418 scales the raw predicted signal based on a minimum and a maximum of experimental signals to create a scaled predicted signal, and at block 516, the squiggle prediction engine 418 adds the scaled predicted signal to the template squiggle. The scaled predicted signal is a scalar value that represents the predicted amplitude of the signal generated by the nanopore at the data point associated with the sliding window location.

At block 518, the squiggle prediction engine 418 determines a distance to move the sliding window for the next window step. In some embodiments, the squiggle prediction engine 418 may perform consistently, in that it may move the sliding window forward a set number of residues for each window step. In some embodiments, the set number of residues may be a single residue. In some embodiments, the set number of residues may be greater than one, in order to coincide with a number of residues expected to move through the nanopore during each sampling interval. For example, it has been experimentally determined that, for a certain combination of nanopore 104 and motor protein, the protein is expected to take an average step size of 2 residues, with a distribution of distances centered around 2 residues. Accordingly, a distance of 2 residues may be a suitable distance to move the sliding window for the next window step. In some embodiments, the distance to move the sliding window may be determined based on characteristics of the residues within or neighboring the sliding window.

In some embodiments, the squiggle prediction engine 418 may introduce an amount of irregularity into the movement of the sliding window, in that the squiggle prediction engine 418 may move the sliding window forward more than one residue, either based on the residues within the sliding window or based on a random determination in order to mimic actual transit of proteins through the nanopore.

The method 500 then advances to for-loop end block 520. If the determination at block 518 has moved the sliding window elsewhere within the sequence, then the method 500 returns to for-loop start block 506 to process the contents of the sliding window after being moved to the location determined by block 518. Otherwise, if the determination at block 518 has moved the sliding window outside of the sequence or another end condition has been detected, then the method 500 proceeds from for-loop end block 520 to an end block and terminates. The end conditions may include reaching the end of the sequence, reaching a maximum length of a read generated by the nanopore, reaching a preconfigured maximum length threshold, or any other suitable condition.

Upon the conclusion of the method 500, a vector of scaled predicted signal values has been determined that represents a template squiggle for the protein. The template squiggle represents a simulation of the transit of the protein through the nanopore, and may be used to train machine learning models to classify proteins as described in further detail below.

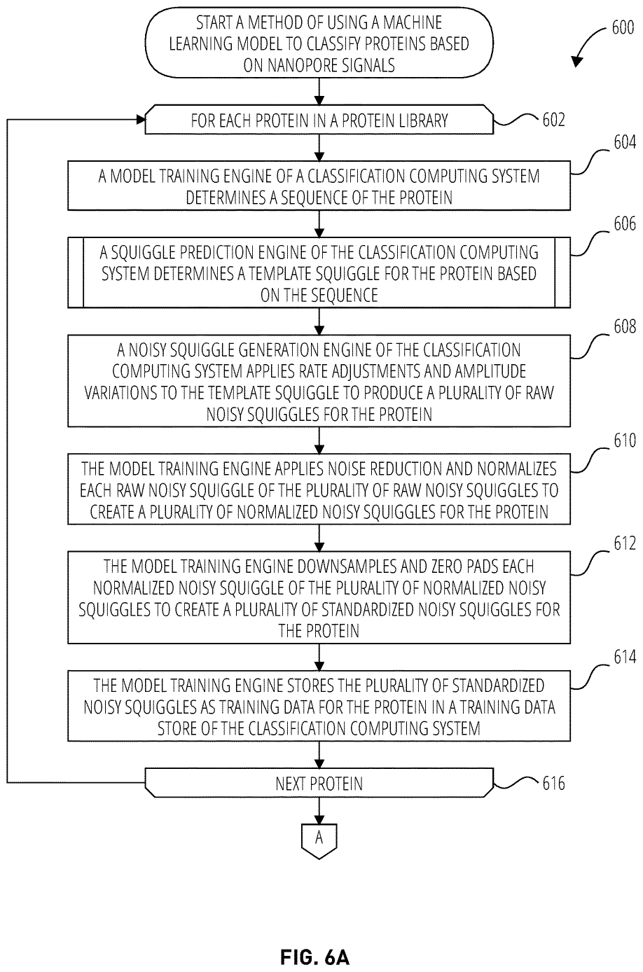

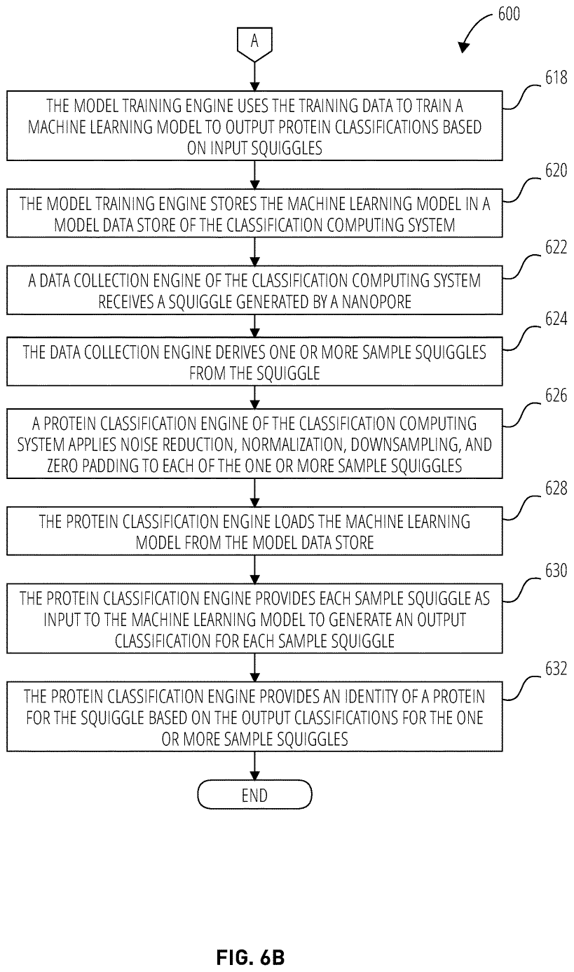

FIG. 6A-FIG. 6B are a flowchart that illustrates a non-limiting example embodiment of a method of using a machine learning model to classify proteins based on nanopore signals, according to various aspects of the present disclosure. As discussed above, the difficulty in the supervised training of machine learning models to classify proteins within the human proteome, including but not limited to artificial neural network models such as convolutional neural network (CNN) models, is that supervised learning typically uses labeled training data that is representative of the expected inputs to the model. Given the size of the human proteome, it is impractical to collect actual training data for most of the protein library, and so the method 600 utilizes template squiggles generated based on the known sequences of the proteins to be detected in order to generate training data.

From a start block, the method 600 proceeds to a for-loop defined between a for-loop start block 602 and a subroutine block 606, wherein each protein in a protein library is processed for the creation of training data. From the for-loop start block 602, the method 600 proceeds to block 604, where a model training engine 420 of a classification computing system 206 determines a sequence of the protein. In some embodiments, the model training engine 420 may retrieve the sequence of the protein from the reference data store 414. In some embodiments, the model training engine 420 may retrieve the sequence of the protein from some other data source, such as a third party data store that makes sequence information available for the human proteome.

The method 600 then advances to subroutine block 606, where a subroutine is executed in which a squiggle prediction engine 418 of the classification computing system 206 determines a template squiggle for the protein based on the sequence. Any suitable subroutine may be used, including but not limited to the method 500 illustrated in FIG. 5 and discussed in detail above.

At block 608, a noisy squiggle generation engine 416 of the classification computing system 206 applies rate adjustments and amplitude variations to the template squiggle to produce a plurality of raw noisy squiggles for the protein.

In some embodiments, the subroutine executed in subroutine block 606 generates an idealized template squiggle, in which it is assumed that the protein moves through the nanopore at a constant rate (i.e., the sliding window is moved forward consistently in steps of a set size). However, in experimental data, it is often found that the protein does not move smoothly through the nanopore. Instead, the protein may pass through faster than expected, may be temporarily blocked and move more slowly than expected, or may move in reverse for one or more sampling intervals. Accordingly, the noisy squiggle generation engine 416 may apply rate adjustments to the template squiggle that insert skips, stalls, and/or changes of direction at random points along the template squiggle to create a non-smooth squiggle that more closely resembles experimental traces. Any suitable frequency and size of random skipping, stalling, and direction reversal may be applied, and may be based on experimental evidence generated for a limited sample set of proteins.

Further, the nanopore is a physical structure as opposed to a theoretical model, and so noise in the experimentally generated signals is unavoidable, though it may not be represented in the template squiggle. Accordingly, the noisy squiggle generation engine 416 adds small amplitude variations sampled from a Gaussian throughout the template squiggle. As with the frequency and size of random skipping and stalling, any suitable amplitude variations may be applied, and may be based on experimental evidence generated for a limited sample set of proteins.

The rate adjustments and amplitude variations may be applied randomly, such that each raw noisy squiggle of the plurality of raw noisy squiggles is slightly different from the template squiggle and slightly different from each other, much in the way actual experimental data would be unlikely to exactly match due to random variation. Any number of raw noisy squiggles may be generated for the plurality of raw noisy squiggles, with larger numbers of raw noisy squiggles leading to a greater amount of training data and an associated change in performance of the trained CNN, as discussed in further detail below.

The resulting raw noisy squiggles are typically each an array of a length of about 15,000 entries to about 20,000 entries, depending on the random nature of the applied rate adjustments. While the plurality of raw noisy squiggles may closely resemble raw sample squiggles received from the nanopore, performance of the CNN may be improved by pre-processing the raw sample squiggles prior to submission to the CNN. As such, similar pre-processing may be applied to the raw noisy squiggles prior to being added to the set of training data. Accordingly, at block 610, the model training engine 420 applies noise reduction and normalizes each raw noisy squiggle of the plurality of raw noisy squiggles to create a plurality of normalized noisy squiggles for the protein. Any suitable noise reduction technique may be used, including but not limited to applying a Bessel filter. Any suitable normalization technique may be used as well, including but not limited to Z-score normalization or median-based normalization.

At block 612, the model training engine 420 downsamples and zero pads each normalized noisy squiggle of the plurality of normalized noisy squiggles to create a plurality of standardized noisy squiggles for the protein. Downsampling may be beneficial to improve the ease of computation, as an array of 15,000-20,000 entries may be cumbersome to process. It has been determined that downsampling by a factor of 10% does not unduly impact the accuracy of the trained machine learning model, but it does significantly increase the speed of computation. Accordingly, the normalized noisy squiggles of the plurality of normalized noisy squiggles may be downsampled by a constant amount such that all of the normalized noisy squiggles are less than a threshold length. Any suitable threshold length may be used, including but not limited to threshold lengths in a range of 3,500-4,500, such as 4000. In order to provide fixed-length inputs into the machine learning model, these downsampled normalized noisy squiggles are then zero padded back up to the threshold length to create the plurality of standardized noisy squiggles.

At block 614, the model training engine 420 stores the plurality of standardized noisy squiggles as training data for the protein in a training data store 408 of the classification computing system 206. The method 600 then advances to a for-loop end block 616. If further proteins remain to be processed in the protein library, then the method 600 returns from for-loop end block 616 to for-loop start block 602 to process the next protein in the protein library. Otherwise, if all of the proteins in the protein library have been processed, then the method 600 advances from for-loop end block 616 to a continuation terminal (“terminal A”).

From terminal A (FIG. 6B), the method 600 proceeds to block 618, where the model training engine 420 uses the training data to train a machine learning model to output protein classifications based on input squiggles, and at block 620, the model training engine 420 stores the machine learning model in a model data store 412 of the classification computing system 206. Any suitable architecture may be used for the machine learning model, including but not limited to artificial neural networks such as Convolutional Neural Networks (CNNs). One non-limiting example embodiment of a CNN architecture suitable for use with the method 600 is illustrated in the following table:

```
class CNN(nn.Module):
  def_{——}init_{——}(self):
    super( )._{——}init_{——}( )
    #Input has 1 channel, 8 output channels, kernel_size=15
    self.conv1 = nn.Conv1d(1, 8, 15)
    # Max pooling with kernel_size=2 and stride=2
    self.pool = nn.MaxPool1d(2)
    # Input has 8 channels from previous layer, 16 output channels,
    # kernel_size =15
    self. conv2 = nn.Conv1d(8, 16, 15)
    # Calculate the flattened input size after conv2
    self.fc_input_size = self.calculate_fc_input_size(downsampled_size)
    # Flattened input size after conv2 , 120 output features
    self.fc1 = nn.Linear(self.fc_input_size, 120)
    # 120 input features , 84 output features
    self.fc2 = nn.Linear (120, 84)
    # 84 input features , 8 output features ( num_classes)
    self.fc3 = nn.Linear (84 , 8)
def forward (self, x):
  x = self.pool( F.relu (self.conv1(x)))
  x = self.pool(F.relu (self.conv2(x)))
  x = torch.flatten (x, 1) # Flattens all dimensions except batch
  x = F.relu(self.fc1(x))
  x = F.relu(self.fc2(x))
  x = self.fc3(x)
  return x
```

The machine learning model may be trained using any suitable technique, including but not limited to gradient descent or using an Adam optimizer. Hyperparameters that may be adjusted during training include one or more of an amount of training data generated for each protein, a number of epochs, a momentum, a learning rate, or regression. A description of hyperparameter tuning during training of a non-limiting example embodiment of a CNN is provided below.

At block 622, a data collection engine 410 of the classification computing system 206 receives a squiggle generated by a nanopore. The squiggle is based on a signal generated by a nanopore upon transit of a sample protein through the nanopore, as opposed to a predicted or simulated signal or squiggle. In some embodiments, the squiggle may be based on data received by the data collection engine 410 directly from the nanopore or flow cell. In some embodiments, the squiggle may be retrieved by the data collection engine 410 from a data store.

At block 624, the data collection engine 410 derives one or more sample squiggles from the squiggle. In some embodiments, a portion of the sequence of the sample protein may cause the sample protein to skip backwards through the nanopore compared to its normal direction of transit. This may cause the squiggle to contain multiple reads of some, if not all, of the sample protein. Accordingly, the data collection engine 410 may break apart the multiple reads in the squiggle to form multiple read squiggles from the single squiggle generated by the nanopore. The data collection engine 410 may search for portions of the squiggle that include signal characteristics associated with the backwards motion, and may use these portions of the squiggle as locations to break the squiggle into multiple read squiggles. One type of signal characteristic that may be used is the signal having an amplitude below a skip signal threshold for greater than a threshold amount of time, though other types of signal characteristics may be used.

Once the squiggle is broken into multiple read squiggles, the data collection engine 410 may either provide each of the read squiggles as a sample squiggle for the remainder of the method 600, or may combine the read squiggles into a single sample squiggle. Any suitable technique may be used to combine the read squiggles, including but not limited to averaging, hidden Markov models, Dynamic Time Warping (DTW) averaging, or other techniques. In some embodiments, read squiggles that constitute more than a threshold percentage of the entire sequence, or read squiggles that include a known signal for a beginning of the sequence or other landmark, may be combined, with other read squiggles being discarded. In some embodiments, even read squiggles that cover a small portion of the sequence may be combined to create the sample squiggle.

One will recognize that in some embodiments, the protein may not include a sequence of residues that cause the sample protein to skip backwards through the nanopore. In such cases, the entirety of the squiggle may be used as the sample squiggle.

At block 626, a protein classification engine 422 of the classification computing system 206 applies noise reduction, normalization, downsampling, and zero padding to each of the one or more sample squiggles. The actions of block 626 are similar to those described in blocks 610 and 612, and are intended to put the sample squiggles in a similar format to the standardized noisy squiggles used as training data. In some embodiments, normalization of the sample squiggles may also include normalizing to a known portion of the signal. For example, the sample protein used to generate the squiggle may include an adapter sequence or other landmark that should produce a predictable signal. The entire squiggle may be normalized to adjust the known portion of the signal to be within desired bounds. Though illustrated as being performed after block 624, in some embodiments, the actions of block 626 may be applied to the squiggle prior to the derivation of the sample squiggles at block 624.

At block 628, the protein classification engine 422 loads the machine learning model from the model data store 412, and at block 630, the protein classification engine 422 provides each sample squiggle as input to the machine learning model to generate an output classification for each sample squiggle. In some embodiments, the output classification generated by the machine learning model includes a confidence value.

At block 632, the protein classification engine 422 provides an identity of the protein for the squiggle based on the output classifications for the one or more sample squiggles. If a single sample squiggle was derived at block 624, then the single output classification may be used as the identity of the protein. If multiple sample squiggles were derived at block 624, then the output classifications may be combined in any suitable way to determine the identity of the protein. For example, an output classification having a highest confidence value may be used as the identity of the protein. As another example, each of the output classifications may be considered a “vote,” and the output classification receiving the most votes may be used as the identity of the protein. The method 600 then proceeds to an end block and terminates.

Embodiments of the method 600 were tested using a protein library that included 8 synthetic proteins having sequences that were determined to be distinct and compatible with classification. 672 labeled sample squiggles were obtained for the 8 synthetic proteins, and were used as test data for the trained models. Initially, the CNN model described above was used with no optimizations or improvements, and was trained with 100 standardized noisy squiggles in the training data set for each of the 8 synthetic proteins. When training the CNN model using noisy squiggles as both a training set and a test set, the initial CNN achieved 64% accuracy, which clearly outperforms random guessing. When testing the trained model against the 672 labeled sample squiggles, the accuracy of the initial CNN was 17%, which was consistent across multiple runs and also better than random chance.

Various adjustments and optimizations were made to the CNN to attempt to improve performance against the 672 labeled sample squiggles. In one attempt, the number of standardized noisy squiggles in the training set for each protein was adjusted, with the following results:

| # Noisy Squiggles/ | | |
|---|---|---|
| protein | Generated Accuracy | Experimental Accuracy |
| 100 | 58.3% | 17.4% |
| 500 | 79.8% | 12.4% |
| 1000 | 92.4% | 16.7% |

Increasing the number of noisy squiggles resulted in an increase in the accuracy of the classification of test sets pulled from the noisy squiggles, but did not significantly change the accuracy when classifying the sample squiggles. This indicates that while there are features within the squiggles that can be recognized by a CNN, the increase of training data results in overfitting to the training set, and does not generalize to the sample squiggles. It may also indicate there is a distinct difference between the noisy squiggles and the sample squiggles that is recognized by the CNN.

In another attempt, the number of epochs for training the CNN was adjusted. The number of epochs is the number of times the training data set is pushed through the CNN during training. This hyperparameter may be highly coupled to other parameters, specifically the amount of training data. A CNN trained with less training data does not see a large sample space for the data, and so it would be expected for more epochs to be used to converge to a local minimum in the loss function. Adjusting the number of epochs often balances learning with overfitting. To test this hyperparameter, the CNN was trained for each of 100, 500, and 1000 noisy squiggles of training data for each protein, and the performance was evaluated with a validation set along equally spaced intervals during training. The results were highly inconsistent run-to-run, but did indicate that experimental accuracy rises and peaks at a certain number of epochs, and beyond that it declines slowly. This technique attempts to find the best model for a given image size. To account for variance along epochs, for each run performance is evaluated along epochs. The experimental accuracy is determined to be the maximum accuracy found across validation intervals. In essence, this change allows the highly coupled relationship between epochs and other hyperparameters to be ignored: by choosing the best, other hyperparameters may be tuned without worrying about how this change affects the overfitting threshold. The best value is simply taken, ensuring that with those other criteria fixed, some epoch level can create the accuracy.

Momentum and learning rate were also adjusted. Momentum is a parameter in gradient descent optimization that builds inertia along the path towards a local minimum, with the goal to overcome weak local minima and avoid infinite oscillation across noisy gradients. Learning rate is a parameter in gradient descent optimization that determines the step size at each iteration when moving in the direction to minimize a loss function. The initial values for momentum and learning rate were 0.15 and 0.01, respectively. To determine an optimal pair of momentum and learning rate values, a standard grid search was performed, training the CNN with these hyperparameters adjusted and leaving everything else unchanged. The results were as follows:

| | Momentum | | | | |
|---|---|---|---|---|---|
| Learning Rate | 0.15 | 0.25 | 0.35 | 0.45 | 0.55 |
| 5e⁻⁴ | 16.1% | 12.8% | 19.6% | 21.7% | 21.6% |
| 1e⁻³ | 16.8% | 22.6% | 21.6% | 19.8% | 16.7% |
| 5e^{-e} | 21.3% | 18.0% | 25.0% | 17.4% | 16.2% |
| 1e⁻² | 19.5% | 17.7% | 20.2% | 19.3% | 17.7% |
| 5e⁻² | 19.6% | 12.2% | 19.6% | 19.6% | 19.6% |
| 1e⁻² | 19.6% | 10.1% | 14.7% | 13.2% | 19.6% |

There was a notable difference obtained in experimental accuracy by changing these hyperparameters. Specifically, the best model was found in the middle of the range for momentum and learning rate, consistently performing over 20%. Due to the variation among individual runs, anything around the middle was not within statistical significance.

Another common hyperparameter to tune is adding regression to the model. Suppose we are given X∈ [symbol] and we wish to predict Y∈ [symbol] if (X, Y)−P_{XY}. Denote

$$\hat{f} = argmin \frac{1}{n} {\sum }_{i = 1}^{n} {( {y}_{i} - f ( {x}_{i} ) )}^{2} .$$

Written mathematically, the machine learning task is to find a function η that minimizes

$$\begin{matrix}\text{?} [ {( Y - η ( X ) )}^{2} ] & = \text{?} [ \text{?} [ {( Y - η ( x ) )}^{2} \vert X = x ] ] \\ & = \text{?} [ {( Y - η ( x ) )}^{2} \vert X = x ] + \text{?} [ ( η ( x ) - {\hat{f}}_{D} ( x ) ) 2 ] \\ & = \underbrace{\text{?} [ {( Y - η ( x ) )}^{2} \vert X = x ]} + \underbrace{{( η ( x ) - \text{?} [ {\hat{f}}_{D} ( x ) ] )}^{2}} + \underbrace{\text{?} [ {( \text{?} [ {\hat{f}}_{D} ( x ) ] - {\hat{f}}_{D} ( x ) )}^{2} ]}\end{matrix}$$

wherein the first bracketed term is irreducible error, the second bracketed term is bias squared, and the third bracketed term is variance. As learning error can be split into bias and variance, and irreducible error is implicit in a model, there exists a bias-variance tradeoff in a machine learning model. The idea behind regression is to add an implicit penalty to the loss function, intentionally increasing bias to try to reduce variance in the model in the hope of reducing the overall learning error of the model.

To this end, L₁ and L₂ regression were performed using the norms ∥ ∥₁ and ∥ ∥₂, respectively. The results were as follows:

| Regularization | L₁ | L₂ |
|---|---|---|
| 5e⁻⁵ | 24.1% | 21.4% |
| 1e⁻⁴ | 23.3% | 18.8% |
| 5e⁻⁴ | 20.8% | 19.3% |
| 1e⁻³ | 27.5% | 18.3% |
| 5e⁻³ | 19.8% | 22.8% |
| 1e⁻² | 19.5% | 18.2% |
| 5e⁻² | 22.9% | 19.6% |
| 1e⁻¹ | 24.1% | 19.6% |
| 5e⁻¹ | 26.0% | 19.6% |

Though arguably not large, there were some significant changes to the model by adding regression. We found that L₁ generally improved accuracy as compared to L₂, but the differences were minimal. Combining this approach with the momentum and epoch pair, the total accuracy achieved was about 27.5% on sample squiggles.

The CNN was also investigated using Dynamic Time Warping (DTW) scores. In the sample squiggles, each sample squiggle is labeled with a DTW score measuring a distance between the sample squiggle and the associated template squiggle. It was found that the CNN has higher levels of accuracy for sample squiggles having DTW scores in the lowest 20th percentile (as high as about 35%), with lower levels of accuracy for sample squiggles having DTW scores in the highest percentiles (as low as 27%).

Other adjustments were also made to the training pipeline. For example, in one test, the sample squiggles were used to train the CNN instead of the generated training data, and the CNN was tested on the generated training data. This provided an accuracy of 22.5%. In another test, the CNN was trained and tested on the sample squiggles alone. This provided an accuracy in classifying the sample squiggles of 33.3%. While this outperformed any experimental accuracy, it should be noted that one of the problems to be overcome by embodiments of the present disclosure is the unavailability of sample squiggles for large portions of the human proteome, and so this value can be thought of as a theoretical upper bound for the CNN model. The accuracy numbers reported above reflect a global accuracy across all eight tested synthetic proteins.

Upon individual analysis of the tested synthetic proteins, it was found that certain proteins were more easily distinguishable by the CNN model than others. Since the output of the CNN may be a probability value for each class, in some embodiments, accuracy may be measured by considering whether the correct value is in the top-N classifications provided by the CNN model, as opposed to the top-1 classification as measured above. When expanding the consideration to top-2, the accuracy increased from about 20% to almost 40%. When expanding to top-3, the accuracy increased to about 55%. These results may be particularly helpful when adapting from the eight synthetic proteins to the full human proteome—a probability distribution over 20,000 proteins will likely not have a large margin between top-1 and top-2, and so allowing N to vary as a hyperparameter for accuracy may provide a more reasonable way of assessing accuracy of a large-scale model.

Machine learning models such as the CNN model described above are fairly complex, and other, simpler types of models may also provide accurate classification results by directly using template squiggles for training instead of using the template squiggles to generate a large volume of training data. For example, a nearest-neighbor model is considerably simpler than a CNN. A nearest-neighbor model includes a set of data points and a distance metric to represent how far data points are from each other. The model is trained by adding a plurality of labeled data points (e.g., template squiggles) to the model. When a new data point (e.g., a sample squiggle) to be classified is received, the distance metric is used to find the k nearest neighbors of the new data point, with the lowest distances indicating the nearest neighbors. Each of the k neighbors casts a vote for the class to which the neighbor belongs as being the correct classification, and the class having a plurality of votes is provided as the classification. The value of k is a hyperparameter that may be adjusted in different circumstances.

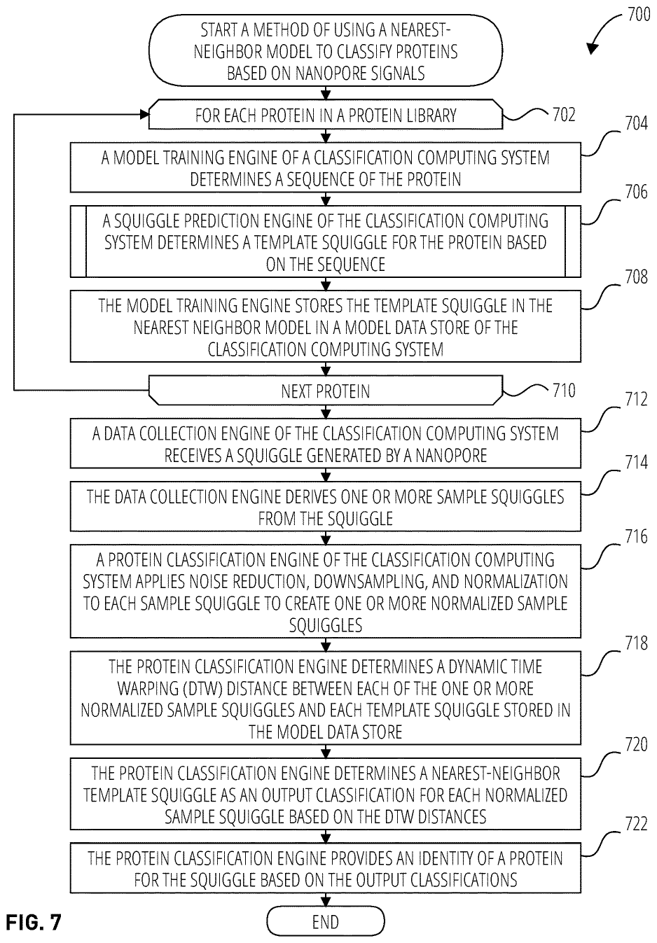

FIG. 7 is a flowchart that illustrates a non-limiting example embodiment of a method of using a nearest-neighbor model to classify proteins based on nanopore signals, according to various aspects of the present disclosure. In the illustrated embodiment, a nearest-neighbor model with k=1 is used. In other words, the single nearest data point stored in the model is used as the classification for the sample squiggle.

From a start block, the method 700 proceeds to a for-loop defined between a for-loop start block 702 and a for-loop end block 710, wherein each protein in a protein library is processed for the creation of training data. From the for-loop start block 702, the method 700 proceeds to block 704, where a model training engine 420 of a classification computing system 206 determines a sequence of the protein. As with the method 600 discussed above, the model training engine 420 may retrieve the sequence of the protein from a reference data store 414 of the classification computing system 206, or from a third party data source for protein sequence information.

The method 700 then advances to subroutine block 706, where a subroutine is executed in which a squiggle prediction engine 418 of the classification computing system 206 determines a template squiggle for the protein based on the sequence. Again, any suitable technique may be used to generate the template squiggle, including but not limited to the method 500 illustrated in FIG. 5 and discussed in detail above.

At block 708, the model training engine 420 stores the template squiggle in the nearest-neighbor model in a model data store 412 of the classification computing system 206. The template squiggle is stored along with a label identifying the associated protein from the protein library. In contrast to the method 600 for training a CNN, this method 700 does not store a plurality of squiggles based on the template squiggle as training data, but instead stores the template squiggle itself in the nearest-neighbor model as a data point. This leads to a reduction in processing time, as well as a reduction in the storage space used within the model data store 412. Though not illustrated in FIG. 7, in some embodiments, the model training engine 420 may apply one or more of noise reduction (e.g, a Bessel filter), normalization, or downsampling as discussed in the method 600 prior to storing the template squiggle in the nearest-neighbor model. Unlike the method 600, the method 700 may not apply zero padding, because the distance metric used in the nearest-neighbor model may be chosen to accommodate comparisons between squiggles of different lengths.

The method 700 then proceeds to the for-loop end block 710. If any further proteins remain in the protein library to be processed, then the method 700 returns to for-loop start block 702 to process the next protein. Otherwise, if all of the proteins in the protein library have been processed, then the method 700 proceeds from for-loop end block 710 to block 712.

At block 712, a data collection engine 410 of the classification computing system 206 receives a squiggle generated by a nanopore. At block 714, the data collection engine 410 derives one or more sample squiggles from the squiggle. The receipt of the squiggle and the derivation of sample squiggles is similar to blocks 622 and 624, and so is not described again here for the sake of brevity.

At block 716, a protein classification engine 422 of the classification computing system 206 applies noise reduction, normalization, and downsampling to each sample squiggle to create one or more normalized sample squiggles. In some embodiments, one or more of these actions may not be performed, either on the sample squiggles or the template squiggles. While it may be desirable to have the same set of actions applied to the sample squiggles as to the template squiggle, in some embodiments, the template squiggles may be relatively noise free, may be generated at a lower data rate than the sample squiggle, and may already be normalized during its creation in the subroutine block 706. In such cases, the actions may not be applied to the template squiggles.

At block 718, the protein classification engine 422 determines a dynamic time warping (DTW) distance between each of the one or more normalized sample squiggles and each template squiggle stored in the model data store 412. DTW is a technique known to those of skill in the art of time series analysis for determining an amount of similarity between two sequences of values that may have been generated at different rates.

At block 720, the protein classification engine 422 determines a nearest-neighbor template squiggle as an output classification for each normalized sample squiggle based on the DTW distances. If a normalized sample squiggle is highly similar to a given template squiggle, then the DTW distance will be relatively low. If a normalized sample squiggle is highly different from a given template squiggle, then the DTW distance will be relatively high. Accordingly, a nearest-neighbor template squiggle for a given normalized sample squiggle may be chosen as the output classification based on the template squiggle having the lowest DTW distance.

At block 722, the protein classification engine 422 provides an identity of a protein for the squiggle based on the output classifications. Any suitable technique may be used to choose a protein if more than one output classification is present. For example, the output classification associated with the nearest-neighbor template squiggle that is closest to any of the normalized template squiggles (the lowest of all of the DTW distances) may be chosen as the identity of the protein. As another example, the output classification associated with the nearest-neighbor template squiggle having the most “votes” (that is, the nearest-neighbor template squiggle that is considered the nearest-neighbor to the highest number of the normalized sample squiggles) may be chosen as the identity of the protein. The method 700 then proceeds to an end block and terminates.

Upon testing, the nearest-neighbor model significantly outperformed the CNN model. The unoptimized version of the nearest-neighbor model provided an accuracy of 60.38% in identifying a test set of noisy squiggles, and an accuracy of 54.61% in identifying the sample squiggles (compared to a peak of 27.5% for the CNN model). Not only did the accuracy of the nearest-neighbor model in identifying the sample squiggles vastly outperform the peak performance of the CNN model, it also provides certain benefits in computing speed as well. While the CNN uses a linear process to analyze its training data while it tunes weights and biases, the nearest-neighbor model does not. It simply compares a sample squiggle with the stored collection of template squiggles. Both the generation of template squiggles and the comparison of sample squiggles to template squiggles are embarrassingly parallel, and can easily be distributed amongst multiple processing cores using multithreaded architectures already present in programming environments such as Python. For example, while the nearest-neighbor model used 1 hr 17 min of run time without the use of parallelization, using 16 cores to parallelize the processing of the nearest-neighbor model reduced the run time to 8 min, which is about a 90% decrease in processing time.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern.

The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, 10 when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims.

As used herein, “nucleic acid” refers to a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxymethylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA. The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyrobo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

As used herein, “peptide” refers to refers to natural biological or artificially manufactured short chains of amino acid monomers linked by peptide (amide) bonds. As used herein, a peptide has at least 2 amino acid repeating units.

As used herein, “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

As used herein, “protein” refers to any of various naturally occurring substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, “tissue” refers to an aggregate of similar cells and cell products forming a definite kind of structural material with a specific function, in a multicellular organism.

### EXAMPLES

The following numbered examples describe non-limiting example embodiments of the disclosed subject matter.

Example 1. A computer-implemented method of training a classifier model for classifying a protein as a specific protein from a protein library, the method comprising: generating, by a computing system, a template squiggle for each protein in the protein library; training, by the computing system, a classifier model based on the template squiggles; and storing, by the computing system, the classifier model in a model data store.

Example 2. The computer-implemented method of Example 1, further comprising: receiving, by the computing system, a squiggle representing a signal generated by passing the protein through a nanopore; and classifying the protein by using the trained classifier model to classify the squiggle.

Example 3. The computer-implemented method of Example 2, further comprising: breaking, by the computing system, the squiggle into one or more read squiggles; and deriving, by the computing system, one or more sample squiggles from the one or more read squiggles; wherein classifying the protein by using the trained classifier model to classify the squiggle includes: providing the one or more sample squiggles as input to the trained classifier model to generate one or more output classifications; and classifying the protein based on the one or more output classifications.

Example 4. The computer-implemented method of any one of Examples 1-3, wherein each protein in the protein library is associated with a known sequence of residues; and wherein generating the template squiggle for each protein in the protein library includes, for each protein in the protein library: determining a combined volume and charge value for each residue in the sequence of residues; determining an initial position of a sliding window within the sequence of residues; determining a predicted signal value for the initial position of the sliding window by: applying a weight to each combined volume and charge value to create adjusted combined volume and charge values; and combining the adjusted combined volume and charge values to create a raw predicted signal value for the initial position of the sliding window.

Example 5. The computer-implemented method of Example 4, wherein determining the predicted signal value for the initial position of the sliding window further includes scaling the raw predicted signal value based on a minimum and a maximum of experimental signals.

Example 6. The computer-implemented method of any one of Examples 4-5, wherein generating the template squiggle for each protein in the protein library further includes, for each protein in the protein library: determining a direction to move the sliding window from the initial position of the sliding window to a new position of the sliding window; and determining a predicted signal value for the new position of the sliding window.

Example 7. The computer-implemented method of any one of Examples 4-6, wherein a size of the sliding window is 20 residues.

Example 8. The computer-implemented method of any one of Examples 4-7, wherein applying the weight to each combined volume and charge value includes applying a negative parabolic weight to the combined volume and charge values.

Example 9. The computer-implemented method of any one of Examples 4-8, wherein for at least one protein in the protein library, the sequence of residues includes at least one post-translational modification.

Example 10. The computer-implemented method of any one of Examples 1-9, wherein the classifier model is a convolutional neural network; and wherein training the classifier model based on the template squiggles includes: for each template squiggle: generating a plurality of noisy squiggles based on the template squiggle; and adding the plurality of noisy squiggles to a set of training data; and training the convolutional neural network based on the set of training data.

Example 11. The computer-implemented method of Example 10, wherein generating the plurality of noisy squiggles includes: applying rate adjustments and amplitude variations to the template squiggle to produce a plurality of raw noisy squiggles; applying noise reduction and normalization to each raw noisy squiggle of the plurality of raw noisy squiggles to create a plurality of normalized noisy squiggles; and downsampling and zero padding the normalized noisy squiggles of the plurality of normalized noisy squiggles to create a plurality of standardized noisy squiggles.

Example 12. The computer-implemented method of any one of Examples 1-11, wherein the classifier model is a nearest neighbor model; and wherein training the classifier model based on the template squiggles includes storing each template squiggle in the nearest neighbor model.

Example 13. The computer-implemented method of Example 12, further comprising: receiving, by the computing system, a squiggle representing a signal generated by passing the protein through a nanopore; and classifying the protein by using the trained classifier model to classify the squiggle; wherein classifying the protein by using the trained classifier model to classify the squiggle includes: determining dynamic time warping (DTW) distances between one or more sample squiggles derived from the squiggle and the template squiggles in the nearest neighbor model; and classifying the protein as a protein of the protein library based on the DTW distances.

Example 14. A non-transitory computer-readable medium having computer-executable instructions stored thereon that, in response to execution by one or more processors of a computing system, cause the computing system to perform actions of a method as recited in any one of Examples 1-13.

Example 15. A computing system comprising at least one processor and a computer-readable medium having computer-executable instructions stored thereon that, in response to execution by the at least one processor, cause the computing system to perform actions of a method as recited in any one of Examples 1-13.

Example 16. A computer-implemented method of classifying a protein as a specific protein from a protein library, the method comprising: receiving, by a computing system, a squiggle representing a signal generated by passing the protein through a nanopore; and classifying the protein by using a trained classifier model to classify the squiggle.

Example 17. The computer-implemented method of Example 16, wherein the trained classifier model was trained based on template squiggles generated for each protein in the protein library.

Example 18. The computer-implemented method of any one of Examples 16-17, further comprising: breaking, by the computing system, the squiggle into one or more read squiggles; and deriving, by the computing system, one or more sample squiggles from the one or more read squiggles; wherein classifying the protein by using the trained classifier model to classify the squiggle includes: providing the one or more sample squiggles as input to the trained classifier model to generate one or more output classifications; and classifying the protein based on the one or more output classifications.

Example 19. The computer-implemented method of Example 18, wherein breaking the squiggle into one or more read squiggles includes: breaking the squiggle into the one or more read squiggles based on detected portions of the squiggle that include signal characteristics associated with backwards motion of the protein through the nanopore.

Example 20. The computer-implemented method of any one of Examples 18-19, wherein deriving the one or more sample squiggles from the one or more read squiggles includes one or more of averaging two or more read squiggles, applying hidden Markov models to two or more read squiggles, or applying dynamic time warping averaging to two or more read squiggles.

Example 21. The computer-implemented method of any one of Examples 16-20, wherein the trained classifier model is a nearest neighbor model; and wherein classifying the protein by using the trained classifier model to classify the squiggle includes: determining dynamic time warping (DTW) distances between one or more sample squiggles derived from the squiggle and the template squiggles in the nearest neighbor model; and classifying the protein as a protein of the protein library based on the DTW distances.

Example 22. The computer-implemented method of any one of Examples 16-21, wherein the trained classifier model is a convolutional neural network; and wherein classifying the protein by using the trained classifier model to classify the squiggle includes: providing one or more sample squiggles derived from the squiggle as input to the convolutional neural network to generate one or more output classifications; and determining an identity of the protein for the squiggle based on the output classifications for the one or more sample squiggles.

Example 23. A non-transitory computer-readable medium having computer-executable instructions stored thereon that, in response to execution by one or more processors of a computing system, cause the computing system to perform actions of a method as recited in any one of Examples 16-22.

Example 24. A computing system comprising at least one processor and a computer-readable medium having computer-executable instructions stored thereon that, in response to execution by the at least one processor, cause the computing system to perform actions of a method as recited in any one of Examples 16-22.

1. A computer-implemented method of training a classifier model for classifying a protein as a specific protein from a protein library, the method comprising:
generating, by a computing system, a template squiggle for each protein in the protein library;
training, by the computing system, a classifier model based on the template squiggles; and
storing, by the computing system, the classifier model in a model data store.

2. The computer-implemented method of claim 1, further comprising:
receiving, by the computing system, a squiggle representing a signal generated by passing the protein through a nanopore; and
classifying the protein by using the trained classifier model to classify the squiggle.

3. The computer-implemented method of claim 2, further comprising:
breaking, by the computing system, the squiggle into one or more read squiggles; and
deriving, by the computing system, one or more sample squiggles from the one or more read squiggles;
wherein classifying the protein by using the trained classifier model to classify the squiggle includes:
providing the one or more sample squiggles as input to the trained classifier model to generate one or more output classifications; and
classifying the protein based on the one or more output classifications.

4. The computer-implemented method of claim 1, wherein each protein in the protein library is associated with a known sequence of residues; and
wherein generating the template squiggle for each protein in the protein library includes, for each protein in the protein library:
determining a combined volume and charge value for each residue in the sequence of residues;
determining an initial position of a sliding window within the sequence of residues;
determining a predicted signal value for the initial position of the sliding window by:
applying a weight to each combined volume and charge value to create adjusted combined volume and charge values; and
combining the adjusted combined volume and charge values to create a raw predicted signal value for the initial position of the sliding window.

5. The computer-implemented method of claim 4, wherein determining the predicted signal value for the initial position of the sliding window further includes scaling the raw predicted signal value based on a minimum and a maximum of experimental signals.

6. The computer-implemented method of claim 4, wherein generating the template squiggle for each protein in the protein library further includes, for each protein in the protein library:
determining a direction to move the sliding window from the initial position of the sliding window to a new position of the sliding window; and
determining a predicted signal value for the new position of the sliding window.

7. The computer-implemented method of claim 4, wherein a size of the sliding window is 20 residues.

8. The computer-implemented method of claim 4, wherein applying the weight to each combined volume and charge value includes applying a negative parabolic weight to the combined volume and charge values.

9. The computer-implemented method of claim 4, wherein for at least one protein in the protein library, the sequence of residues includes at least one post-translational modification.

10. The computer-implemented method of claim 1, wherein the classifier model is a convolutional neural network; and
wherein training the classifier model based on the template squiggles includes:
for each template squiggle:
generating a plurality of noisy squiggles based on the template squiggle; and
adding the plurality of noisy squiggles to a set of training data; and
training the convolutional neural network based on the set of training data;
wherein generating the plurality of noisy squiggles includes:
applying rate adjustments and amplitude variations to the template squiggle to produce a plurality of raw noisy squiggles;
applying noise reduction and normalization to each raw noisy squiggle of the plurality of raw noisy squiggles to create a plurality of normalized noisy squiggles; and
downsampling and zero padding the normalized noisy squiggles of the plurality of normalized noisy squiggles to create a plurality of standardized noisy squiggles.

11. (canceled)

12. The computer-implemented method of claim 1, wherein the classifier model is a nearest neighbor model; and
wherein training the classifier model based on the template squiggles includes storing each template squiggle in the nearest neighbor model.

13. The computer-implemented method of claim 12, further comprising:
receiving, by the computing system, a squiggle representing a signal generated by passing the protein through a nanopore; and
classifying the protein by using the trained classifier model to classify the squiggle;
wherein classifying the protein by using the trained classifier model to classify the squiggle includes:
determining dynamic time warping (DTW) distances between one or more sample squiggles derived from the squiggle and the template squiggles in the nearest neighbor model; and
classifying the protein as a protein of the protein library based on the DTW distances.

14. A non-transitory computer-readable medium having computer-executable instructions stored thereon that, in response to execution by one or more processors of a computing system, cause the computing system to perform actions for training a classifier model for classifying a protein as a specific protein from a protein library, the actions comprising:
generating, by a computing system, a template squiggle for each protein in the protein library;
training, by the computing system, a classifier model based on the template squiggles; and
storing, by the computing system, the classifier model in a model data store.

15-24. (canceled)

25. The non-transitory computer-readable medium of claim 14, wherein the actions further comprise:
receiving, by the computing system, a squiggle representing a signal generated by passing the protein through a nanopore;
classifying, by the computing system, the protein by using the trained classifier model to classify the squiggle;
breaking, by the computing system, the squiggle into one or more read squiggles; and
deriving, by the computing system, one or more sample squiggles from the one or more read squiggles;
wherein classifying the protein by using the trained classifier model to classify the squiggle includes:
providing the one or more sample squiggles as input to the trained classifier model to generate one or more output classifications; and
classifying the protein based on the one or more output classifications.

26. The non-transitory computer-readable medium of claim 14, wherein each protein in the protein library is associated with a known sequence of residues; and
wherein generating the template squiggle for each protein in the protein library includes, for each protein in the protein library:
determining a combined volume and charge value for each residue in the sequence of residues;
determining an initial position of a sliding window within the sequence of residues;
determining a predicted signal value for the initial position of the sliding window by:
applying a weight to each combined volume and charge value to create adjusted combined volume and charge values; and
combining the adjusted combined volume and charge values to create a raw predicted signal value for the initial position of the sliding window.

27. The non-transitory computer-readable medium of claim 26, wherein determining the predicted signal value for the initial position of the sliding window further includes scaling the raw predicted signal value based on a minimum and a maximum of experimental signals.

28. The non-transitory computer-readable medium of claim 26, wherein generating the template squiggle for each protein in the protein library further includes, for each protein in the protein library:
determining a direction to move the sliding window from the initial position of the sliding window to a new position of the sliding window; and
determining a predicted signal value for the new position of the sliding window.

29. The non-transitory computer-readable medium of claim 26, wherein applying the weight to each combined volume and charge value includes applying a negative parabolic weight to the combined volume and charge values.

30. The non-transitory computer-readable medium of claim 14, wherein the classifier model is a convolutional neural network; and
wherein training the classifier model based on the template squiggles includes:
for each template squiggle:
generating a plurality of noisy squiggles based on the template squiggle; and
adding the plurality of noisy squiggles to a set of training data; and
training the convolutional neural network based on the set of training data;
wherein generating the plurality of noisy squiggles includes:
applying rate adjustments and amplitude variations to the template squiggle to produce a plurality of raw noisy squiggles;
applying noise reduction and normalization to each raw noisy squiggle of the plurality of raw noisy squiggles to create a plurality of normalized noisy squiggles; and
downsampling and zero padding the normalized noisy squiggles of the plurality of normalized noisy squiggles to create a plurality of standardized noisy squiggles.

31. The non-transitory computer-readable medium of claim 14, wherein the classifier model is a nearest neighbor model;
wherein training the classifier model based on the template squiggles includes storing each template squiggle in the nearest neighbor model; and
wherein the actions further comprise:
receiving, by the computing system, a squiggle representing a signal generated by passing the protein through a nanopore; and
classifying the protein by using the trained classifier model to classify the squiggle;
wherein classifying the protein by using the trained classifier model to classify the squiggle includes:
determining dynamic time warping (DTW) distances between one or more sample squiggles derived from the squiggle and the template squiggles in the nearest neighbor model; and
classifying the protein as a protein of the protein library based on the DTW distances.
